(19) United States
(12) Patent Application Publication
(10) Pub. No.: US 2024/0058112 A1
(43) Pub. Date: Feb. 22, 2024

(54) BODILY IMPLANTS WITH FLUID SYSTEMS

(71) Applicant: Boston Scientific Scimed, Inc., Maple Grove, MN (US)

(72) Inventors: Barbara Belisa Soffiati, Clonmel (IE); Daragh Nolan, Via Youghal (IE); Thomas Sinnott, Wexford (IE); James Michael English, Cahir (IE); Richard Percy, Leamlara (IE); James Shane Power, Dungarvan (IE); Brian P. Watschke, Minneapolis, MN (US); Joseph Prescott, Oakdale, MN (US); Bryan Duane Johnson, Vadnais Heights, MN (US)

(21) Appl. No.: 18/450,556

(22) Filed: Aug. 16, 2023

Related U.S. Application Data
(60) Provisional application No. 63/371,810, filed on Aug. 18, 2022.

(51) Int. Cl. A61F 2/00

(52) U.S. Cl. CPC A61F 2/004; A61F 2250/0058; A61F 2220/0058

(57) ABSTRACT

According to an aspect, an implant, includes an inflatable member, a pump assembly, a tubular member and a coupling member. The pump assembly is configured to facilitate a transfer of fluid to and from the inflatable member. The coupling member is configured to facilitate a coupling of the tubular member to the pump assembly.

### CROSS-REFERENCE TO RELATED APPLICATION

This application claims priority to U.S. Provisional Patent Application No. 63/371,810, filed on Aug. 18, 2022, entitled “BODILY IMPLANTS WITH FLUID SYSTEMS”, the disclosure of which is incorporated by reference herein in its entirety.

### TECHNICAL FIELD

This disclosure relates generally to bodily implants and more specifically to bodily implants that include fluid systems and inflatable members.

### BACKGROUND

Bodily implants with fluid systems and inflatable members may be placed within a body of a patient for a variety of reasons. For example, one treatment for male erectile dysfunction is the implantation of a penile prosthesis that mechanically erects the penis. Additionally, some patients phalloplasty patients may receive a penile prosthesis. In another example, one treatment for urinary incontinence is the implantation of an artificial urinary sphincter. Such penile prosthesis and artificial urinary sphincters may include an inflatable member or members and a fluid system.

### SUMMARY

According to an aspect, an implant, includes an inflatable member, a pump assembly, a tubular member and a coupling member. The pump assembly is configured to facilitate a transfer of fluid to and from the inflatable member. The coupling member is configured to facilitate a coupling of the tubular member to the pump assembly.

In some embodiments, the coupling member includes a first portion disposed within a lumen defined by the tubular member and a second portion disposed within a housing of the pump assembly. In some embodiments, the coupling member includes a first portion disposed within a lumen defined by the tubular member, the first portion of the coupling member having a projection member. In some embodiments, the coupling member includes a first portion disposed within a lumen defined by the tubular member, the first portion of the coupling member having a barb member.

In some embodiments, the pump assembly includes a housing member having a projection, the projection of the housing member being disposed within a lumen defined by the tubular member, the coupling member including a spring biased clip, the coupling member being disposed such that a portion of the tubular member is disposed between the spring biased clip and a portion of the projection. In some embodiments, the pump assembly includes a housing member having a projection, the projection of the housing member being disposed within a lumen defined by the tubular member, the coupling member includes an overlapping wire member. In some embodiments, the pump assembly includes a housing member having a projection, the projection of the housing member being disposed within a lumen defined by the tubular member, the coupling member includes an overlapping wire member, the overlapping wire member including a screw member.

In some embodiments, the coupling member includes a first portion disposed within a lumen defined by the tubular member and a second portion disposed within a housing of the pump assembly, the coupling member defining a lumen and having a filter (or flow modifier) disposed within the lumen defined by the coupling member. In some embodiments, the coupling member includes a first portion disposed within a lumen defined by the tubular member and a second portion disposed within a housing of the pump assembly, the coupling member defining a lumen and having a filter (or flow modifier) disposed within the lumen defined by the coupling member, the filter being a gyroid filter (or having a different shape).

In some embodiments, the coupling member includes a first portion disposed within a lumen defined by the tubular member and a second portion disposed within a housing of the pump assembly, the coupling member defining a lumen and having a filter disposed within the lumen defined by the coupling member, the first portion of the coupling member being laser welded to the housing of the pump assembly.

In some embodiments, the coupling member includes a first portion disposed within a lumen defined by the tubular member, the implant further comprising a form member disposed such that a portion of the tubular member is disposed between the form member and a portion of the coupling member. In some embodiments, the coupling member includes a first portion disposed within a lumen defined by the tubular member, the implant further comprising a form member disposed such that a portion of the tubular member is disposed between the form member and a portion of the coupling member, the form member being disposed within a housing of the pump assembly.

In some embodiments, the tubular member is a first tubular member, the implant further comprising a second tubular member; and a form member defining a first lumen and a second lumen, the first tubular member extending through the first lumen of the form member, the second tubular member extending through the second lumen of the form member.

In some embodiments, the tubular member is a first tubular member, the implant further comprising a second tubular member; and a form member defining a first lumen and a second lumen, the first tubular member extending through the first lumen of the form member, the second tubular member extending through the second lumen of the form member, the form member being disposed within a housing of the pump assembly.

In some embodiments, the tubular member is a first tubular member, the implant further comprising a second tubular member; and a form member defining a first lumen and a second lumen, the first tubular member extending through the first lumen of the form member, the second tubular member extending through the second lumen of the form member, the form member being disposed such that a portion of the first tubular member is disposed between the coupling member and the form member.

In some embodiments, the implant is configured to be placed within a body of a patient. For example, in some embodiments, the implant or device is configured to be placed within a pelvic region of a patient. For example, in some embodiments, the inflatable member may be placed within a pelvic region of the patient. In some embodiments, the implant is a penile implant.

According to another aspect, an implant includes an inflatable member; a reservoir configured to hold fluid; a pump assembly, the pump assembly being configured to facilitate a transfer of the fluid to and from the inflatable member; a first tubular member extending between the inflatable member and the pump assembly; a first coupling member, the first coupling member being configured to facilitate a coupling of the first tubular member to the pump assembly; a second tubular member extending between the reservoir and the pump assembly; and a second coupling member, the second coupling member being configured to facilitate a coupling of the second tubular member to the pump assembly.

In some embodiments, the first coupling member includes a first portion disposed within a lumen defined by the first tubular member and a second portion disposed within a housing of the pump assembly. In some embodiments, the first coupling member includes a first portion disposed within a lumen defined by the first tubular member, the first portion of the first coupling member having a projection member. In some embodiments, the first coupling member includes a first portion disposed within a lumen defined by the first tubular member, the first portion of the first coupling member having a barb member.

In some embodiments, the pump assembly includes a housing member having a projection, the projection of the housing member being disposed within a lumen defined by the first tubular member, the first coupling member including a spring biased clip, the first coupling member being disposed such that a portion of the first tubular member is disposed between the spring biased clip and a portion of the projection.

### BRIEF DESCRIPTION OF THE DRAWINGS

FIG. 1 schematically illustrates an implant according to an embodiment.

FIG. 2 illustrates an implant according to an embodiment.

FIG. 3 illustrates an implant according to an embodiment.

FIG. 4 is a perspective view of a pump assembly according to an embodiment.

FIG. 5 is a side view of a pump assembly according to an embodiment.

FIGS. 6-10 illustrate coupling members according to embodiments.

FIGS. 11-14 illustrate a coupling member according to an embodiment.

FIG. 15 illustrates a filter member according to an embodiment.

FIGS. 16-19 illustrate a pump assembly and a coupling member according to an embodiment.

FIGS. 20-23 illustrate a filter member according to an embodiment.

FIGS. 24-31 illustrate filter members according to embodiments.

FIG. 32 illustrates a coupling member according to an embodiment.

FIGS. 33-37 illustrate a pump assembly according to an embodiment.

FIGS. 38-40 illustrate a pump assembly and a coupling member according to an embodiment.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as penile prostheses, artificial sphincters, or other bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human male, a human female, or any other mammal.

The embodiments discussed herein may improve the performance of a bodily implant. For example, the bodily implant may have improved reliability or improved implantation procedures.

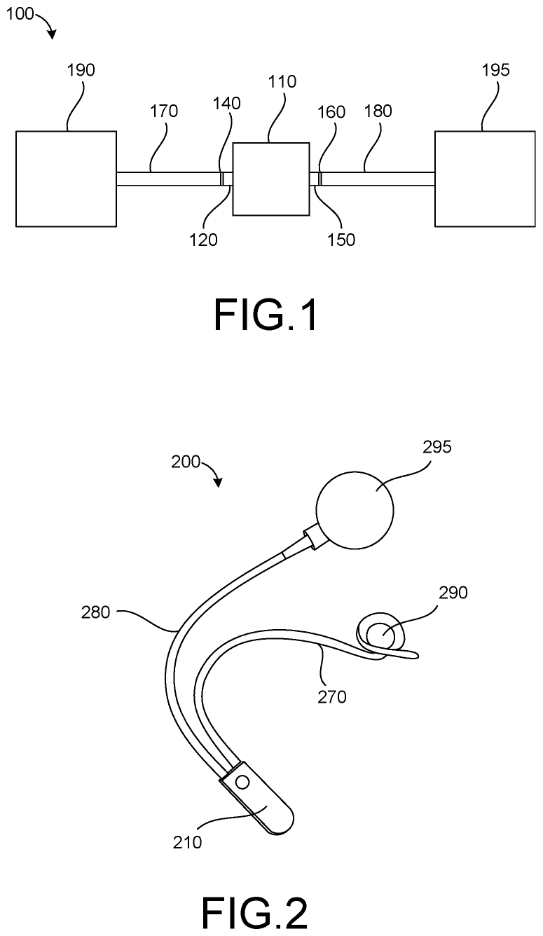

FIG. 1 schematically illustrates a bodily implant 100 according to an embodiment. In some embodiments, the bodily implant 100 may be a penile implant. In other embodiments, the bodily implant 100 may be an artificial urinary sphincter. In yet other embodiments, the bodily implant 100 is a different type of bodily implant.

The illustrated embodiments includes a pump assembly 110, an inflatable member 190, and a reservoir 195. The pump assembly 110 is operatively and fluidically coupled to the pump assembly 110 via tubular member 170. The pump assembly 110 is operatively and fluidically coupled to the reservoir 195 via the tubular member 180.

The reservoir 195 is configured to hold or store a fluid. The pump assembly 110 is configured to move fluid from the reservoir 195 to the inflatable member 190 and from the inflatable member 190 to the reservoir 195. The pump assembly 110 may include electronic pump components or manual pump components. The inflatable member 190 is configured to be placed in an inflated state or configuration and in a deflated state or configuration. In some embodiments, the inflatable member 190 is configured to place pressure on a portion of the body when the inflatable member 190 is in the inflated state or configuration.

In the illustrated embodiment, the tubular member 170 is coupled to the pump assembly 110 and to the inflatable member 190. The tubular member 170 is configured to convey fluid between the pump assembly 110 and the inflatable member 190. The bodily implant 100 includes a coupling member 120. The coupling member 120 is configured to facilitate the coupling of the tubular member 170 to the pump assembly 110. In some embodiments, the coupling member 120 includes a spring biased member or clip. In other embodiments, the coupling member 120 includes an overlapping wire. In yet other embodiments, the coupling member 120 includes a different type of member or component configured to help or facilitate the coupling of the tubular member 170 to the pump assembly 110.

In the illustrated embodiment, the bodily implant 100 includes a filter or a filter member 140. The filter or filter member 140 is disposed between the inflatable member 190 and the pump assembly 110. In some embodiments, the filter or filter member 140 is disposed adjacent or within a portion of the coupling member 120. The filter or filter member 140 is configured to filter out particles or other debris that may be disposed within the fluid. In some embodiments, the filter or filter member 140 is configured to help prevent the particles or other debris from entering the pump assembly 110 or to modify the flow within the pump assembly 110.

In the illustrated embodiment, the tubular member 180 is coupled to the pump assembly 110 and to the reservoir 195. The tubular member 180 is configured to convey fluid between the pump assembly 110 and the reservoir 195. The bodily implant 100 includes a coupling member 150. The coupling member 150 is configured to facilitate the coupling of the tubular member 170 to the pump assembly 110. In some embodiments, the coupling member 150 includes a spring biased member or clip. In other embodiments, the coupling member 150 includes an overlapping wire. In yet other embodiments, the coupling member 150 includes a different type of member or component configured to help or facilitate the coupling of the tubular member 180 to the pump assembly 110.

In the illustrated embodiment, the bodily implant 100 includes a filter or a filter member 160. The filter or filter member 160 is disposed between the reservoir 195 and the pump assembly 110. In some embodiments, the filter or filter member 160 is disposed adjacent or within a portion of the coupling member 150. The filter or filter member 160 is configured to filter out particles or other debris that may be disposed within the fluid. In some embodiments, the filter or filter member 160 is configured to help prevent the particles or other debris from entering the pump assembly 110.

In use, the bodily implant 100 may be placed within the body of a patient. In some embodiments, the inflatable member 190 may be placed such that it is able to place pressure on a portion of the body of the patient when the inflatable member 190 is in the inflated or expanded configuration.

FIG. 2 illustrates a bodily implant 200 according to an embodiment. The implant 200 is an artificial urinary sphincter. The implant 200 includes a pump assembly 210, an inflatable member 290, and a reservoir 295. The pump assembly 210 is operatively and fluidically coupled to the pump assembly 210 via tubular member 270. The pump assembly 210 is operatively and fluidically coupled to the reservoir 295 via the tubular member 280.

The reservoir 295 is configured to hold or store a fluid. The pump assembly 210 is configured to move fluid from the reservoir 295 to the inflatable member 290 and from the inflatable member 290 to the reservoir 295. The pump assembly 210 may include electronic pump components or manual pump components. The inflatable member 290 is configured to be placed in an inflated state or configuration and in a deflated state or configuration. In some embodiments, the inflatable member 290 is configured to place pressure on a portion of the body when the inflatable member 290 is in the inflated state or configuration.

In the illustrated embodiment, the tubular member 270 is coupled to the pump assembly 210 and to the inflatable member 290. The tubular member 270 is configured to convey fluid between the pump assembly 210 and the inflatable member 290. The bodily implant 200 includes a coupling member. The coupling member is configured to facilitate the coupling of the tubular member 270 to the pump assembly 210. In some embodiments, the coupling member includes a spring biased member or clip. In other embodiments, the coupling member includes an overlapping wire. In yet other embodiments, the coupling member includes a different type of member or component configured to help or facilitate the coupling of the tubular member 270 to the pump assembly 210.

In some embodiments, the bodily implant 200 includes a filter or a filter member. The filter or filter member is disposed between the inflatable member 290 and the pump assembly 210. In some embodiments, the filter or filter member is disposed adjacent or within a portion of the coupling member. The filter or filter member is configured to filter out particles or other debris that may be disposed within the fluid. In some embodiments, the filter or filter member is configured to help prevent the particles or other debris from entering the pump assembly 210.

In the illustrated embodiment, the tubular member 280 is coupled to the pump assembly 210 and to the reservoir 295. The tubular member 280 is configured to convey fluid between the pump assembly 210 and the reservoir 295. The bodily implant 200 includes a coupling member. The coupling member is configured to facilitate the coupling of the tubular member 270 to the pump assembly 210. In some embodiments, the coupling member includes a spring biased member or clip. In other embodiments, the coupling member includes an overlapping wire. In yet other embodiments, the coupling member includes a different type of member or component configured to help or facilitate the coupling of the tubular member 280 to the pump assembly 210.

In some embodiments, the bodily implant 200 includes a filter or a filter member. The filter or filter member is disposed between the reservoir 295 and the pump assembly 210. In some embodiments, the filter or filter member is disposed adjacent or within a portion of the coupling member. The filter or filter member is configured to filter out particles or other debris that may be disposed within the fluid. In some embodiments, the filter or filter member is configured to help prevent the particles or other debris from entering the pump assembly 210.

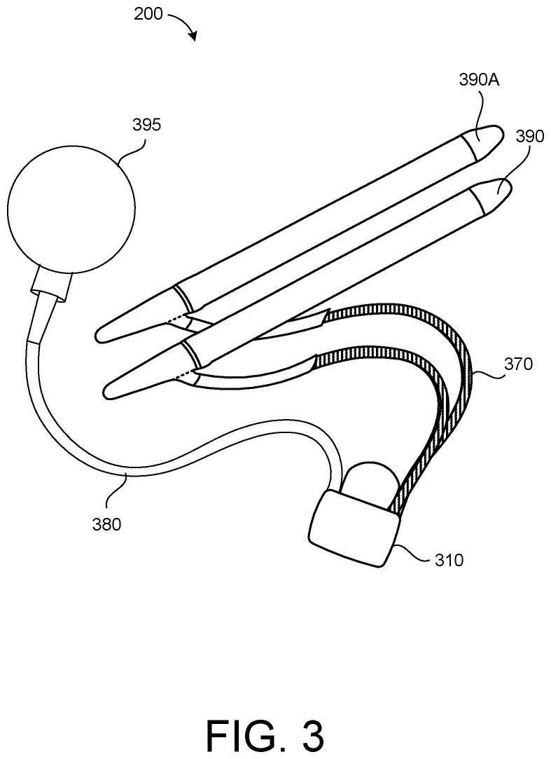

FIG. 3 illustrates a bodily implant 300 according to an embodiment. The implant 300 is penile implant. The implant 300 includes a pump assembly 310, an inflatable member 390, and a reservoir 395. The implant also includes a second inflatable member 390A. The pump assembly 310 is operatively and fluidically coupled to the pump assembly 310 via tubular member 370. The pump assembly 310 is operatively and fluidically coupled to the reservoir 395 via the tubular member 380.

The reservoir 395 is configured to hold or store a fluid. The pump assembly 310 is configured to move fluid from the reservoir 395 to the inflatable member 390 and from the inflatable member 390 to the reservoir 395. The pump assembly 310 may include electronic pump components or manual pump components. The inflatable member 390 is configured to be placed in an inflated state or configuration and in a deflated state or configuration. In some embodiments, the inflatable member 390 is configured to place pressure on a portion of the body when the inflatable member 390 is in the inflated state or configuration.

In the illustrated embodiment, the tubular member 370 is coupled to the pump assembly 310 and to the inflatable member 390. The tubular member 370 is configured to convey fluid between the pump assembly 310 and the inflatable member 390. The bodily implant 300 includes a coupling member. The coupling member is configured to facilitate the coupling of the tubular member 370 to the pump assembly 310. In some embodiments, the coupling member includes a spring biased member or clip. In other embodiments, the coupling member includes an overlapping wire. In yet other embodiments, the coupling member includes a different type of member or component configured to help or facilitate the coupling of the tubular member 370 to the pump assembly 310.

In some embodiments, the bodily implant 300 includes a filter or a filter member. The filter or filter member is disposed between the inflatable member 390 and the pump assembly 310. In some embodiments, the filter or filter member is disposed adjacent or within a portion of the coupling member. The filter or filter member is configured to filter out particles or other debris that may be disposed within the fluid. In some embodiments, the filter or filter member is configured to help prevent the particles or other debris from entering the pump assembly 310.

In the illustrated embodiment, the tubular member 380 is coupled to the pump assembly 310 and to the reservoir 395. The tubular member 380 is configured to convey fluid between the pump assembly 310 and the reservoir 395. The bodily implant 300 includes a coupling member. The coupling member is configured to facilitate the coupling of the tubular member 370 to the pump assembly 310. In some embodiments, the coupling member includes a spring biased member or clip. In other embodiments, the coupling member includes an overlapping wire. In yet other embodiments, the coupling member includes a different type of member or component configured to help or facilitate the coupling of the tubular member 380 to the pump assembly 310.

In some embodiments, the bodily implant 300 includes a filter or a filter member. The filter or filter member is disposed between the reservoir 395 and the pump assembly 310. In some embodiments, the filter or filter member is disposed adjacent or within a portion of the coupling member. The filter or filter member is configured to filter out particles or other debris that may be disposed within the fluid. In some embodiments, the filter or filter member is configured to help prevent the particles or other debris from entering the pump assembly 310.

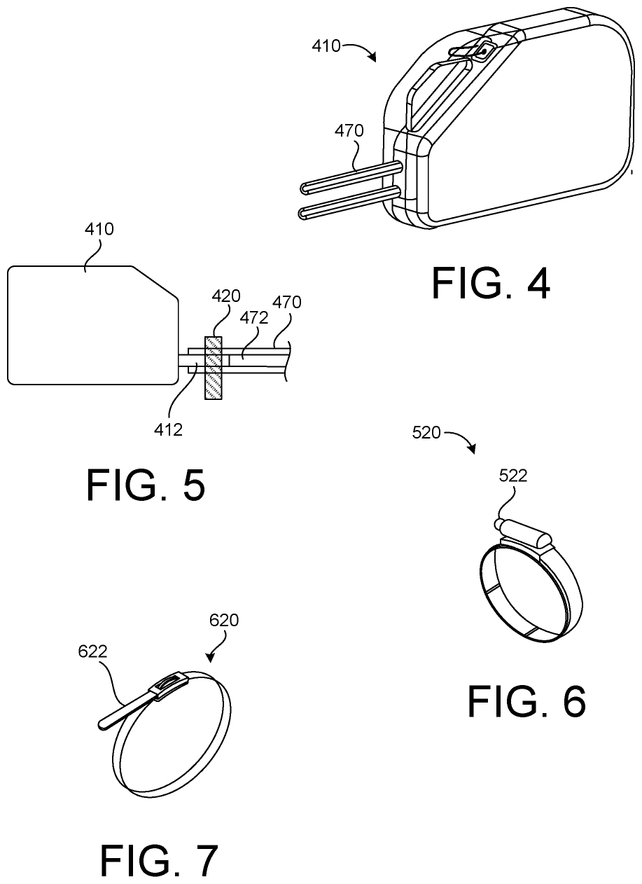

FIG. 4 is a perspective view of a pump assembly 410 according to an embodiment. FIG. 5 is a side view of the pump assembly 410 coupled to a tubular member 470. In the illustrated embodiment, a coupling member 420 couples or help couples the tubular member 470 to the pump assembly 410. The pump assembly 410 includes a projection 412. In some embodiments, the projection 412 defines a lumen. For example, in some embodiments, the projection 412 is a tube or a tubular member. The tubular member 470 defines a lumen 472 and is fit over the projection 412 such that at least a portion of the projection 412 is disposed within the lumen 472 of the tubular member 470. The coupling member 420 is positioned such that a portion of the tubular member 470 is disposed between the coupling member 420 and at least a portion of the projection 412. In some embodiments, the coupling member 420 is configured to place pressure on the tubular member 470 to couple, such as frictionally couple, the tubular member 470 to the pump assembly 410.

FIGS. 6-10 illustrate various embodiments of the coupling member.

As illustrated in FIGS. 6 and 7, in some embodiments, the coupling member 520 and 620 includes a wire that is overlapped with itself. As best illustrated in FIG. 6, the coupling member 520 may include a screw member 522 that is configured to be turned to tighten the coupling member 520 (such as make the diameter of the coupling member 520 smaller). As best illustrated in FIG. 7, in some embodiments, a portion 622 of the coupling member may be pulled to tighten the coupling member 620.

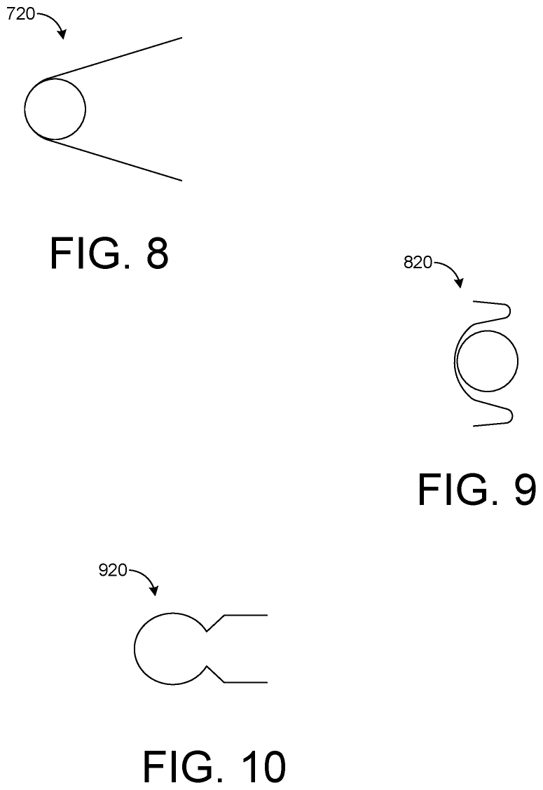

FIGS. 8-10 illustrate biased coupling members 720, 820, and 920. The biased coupling members may be configured to be placed over the tubular member and place pressure on the tubular member to couple the tubular member to the pump assembly. In other embodiments, the biased coupling members may be configured to be placed internally within the tubular member.

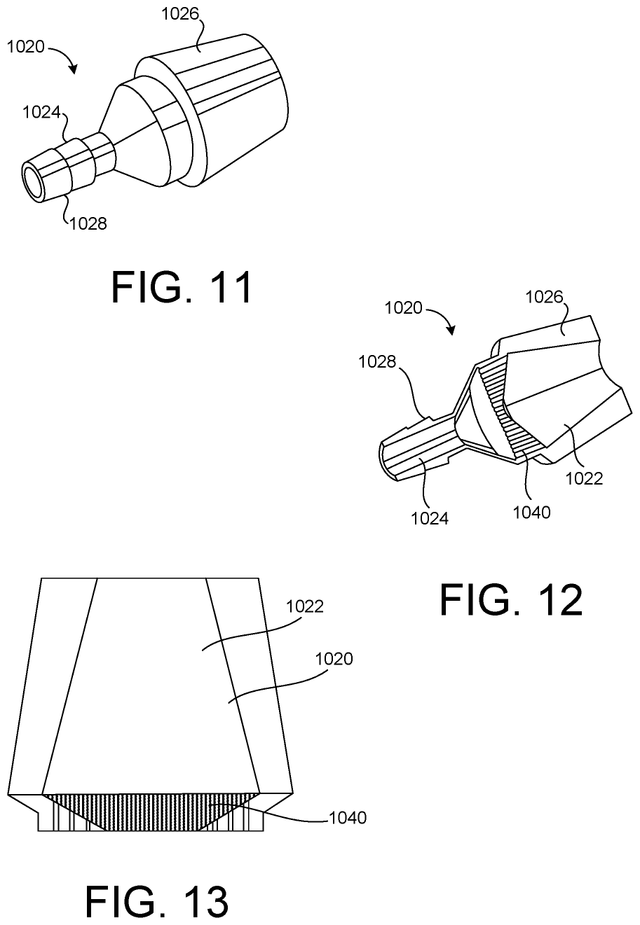

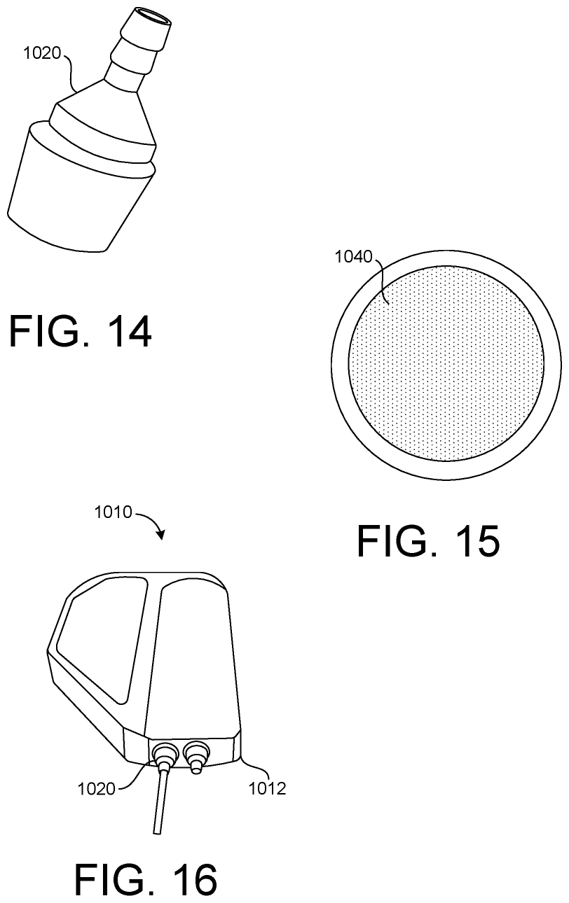

FIGS. 11-15 illustrate a coupling member 1020 according to an embodiment. The coupling member 1020 defines a lumen 1022 and includes a first portion 1024 and a second portion 1026. The first portion 1024 is configured to be coupled to a tubular member. The first portion 1024 includes a ridge, protrusion, or barb 1028. The ridge, protrusion, or barb 1028 is configured to engage the tubular member to help couple the first portion 1024 to the tubular member. In some embodiments, an additional clip or an additional coupling member may be used to secure the coupling member 1020 to the tubular member.

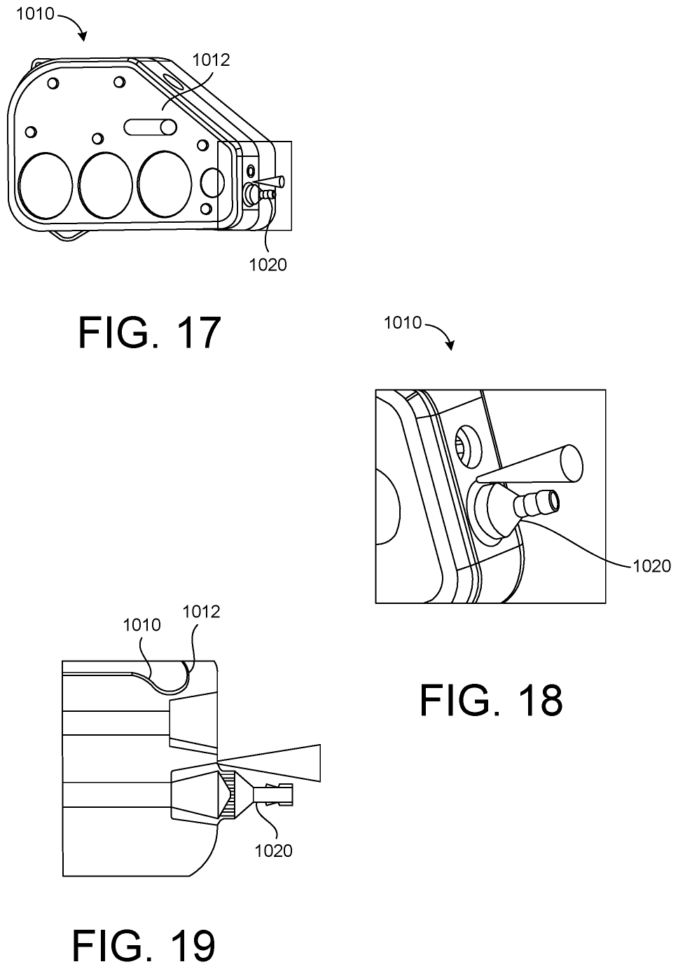

The second portion 1026 is configured to be coupled to a pump assembly. For example, as illustrated in FIGS. 16-19, the second portion 1026 of the coupling member 1020 may be at least partially disposed within in a housing 1012 of a pump assembly 1010. In some embodiments, the second portion 1026 is press fit or frictionally coupled to the housing 1012. In other embodiments, the second portion 1026 is laser welded to the housing 1012. In yet other embodiments, another coupling mechanism, such as an adhesive, may be used to help couple the second portion 1026 to the housing 1012.

The illustrated embodiment, a filter (or flow modifier) 1040 is disposed within the coupling member 1020. The filter 1040 is configured to help prevent particles or debris disposed within the fluid from entering the pump components. In the illustrated embodiment, the filter 1040 is disposed within the lumen 1022 of the coupling member 1020. In some embodiments, the filter 1040 is frictionally coupled within the lumen 1022. In other embodiments, the filter 1040 is laser welded within the lumen 1022. In yet other embodiments, another coupling mechanism is used to couple the filter 1040 within the lumen 1022. In some embodiments, the filter may be formed of several portions or segments coupled together. For example, the several portions or segments may be coupled together via a joining process such as a laser welding process or they may be joined via an adhesive. In some embodiments, the filter and the pump body may be formed or made from a single piece of material. In other words, the filter and the pump body may be unitarily or monolithically formed.

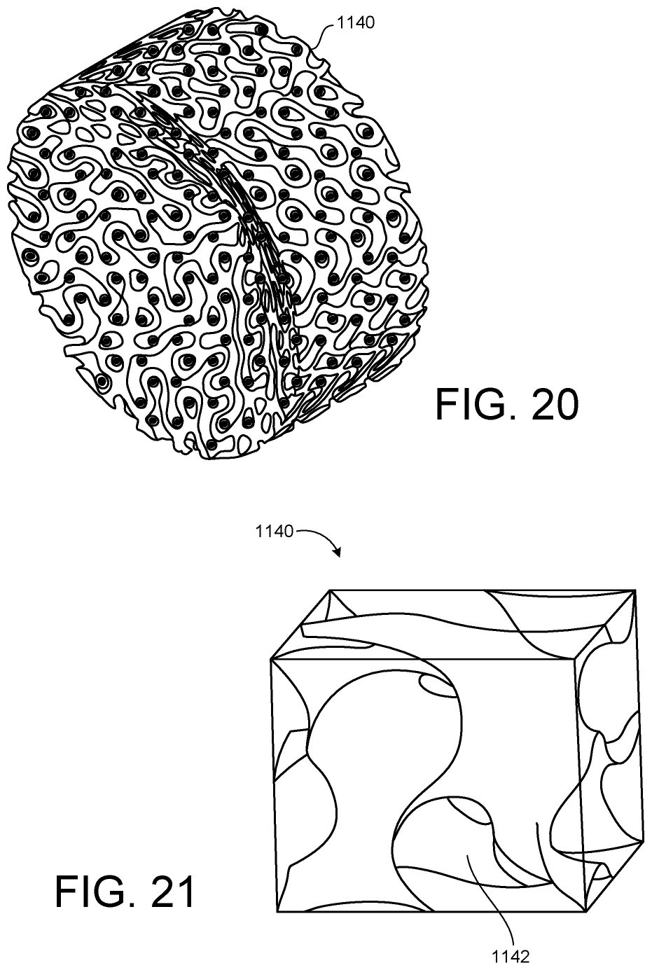

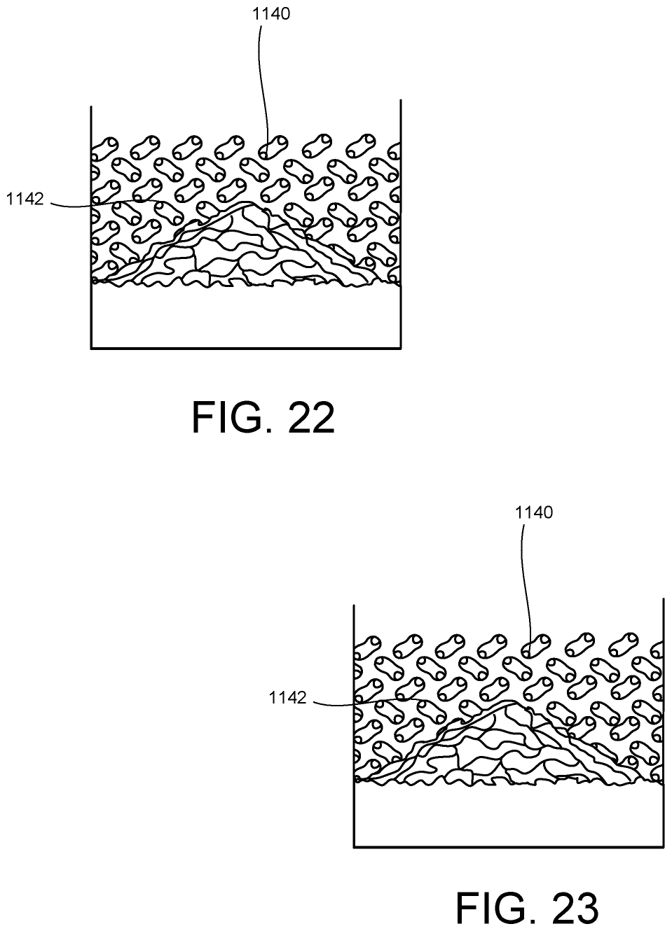

FIGS. 20-23 illustrate a filter 1140 according to an embodiment. The filter 1140 is a gyroid filter. As best illustrated in FIG. 21, the filter 1140 includes or defines lumens 1142. The lumens 1142 extend through the filter 1140 and are non-linear. The lumens 1142 follow a tortuous path as they extend through the filter 1140. FIG. 22 is a front view of the filter 1140. FIG. 23 is a side view of the filter 1140.

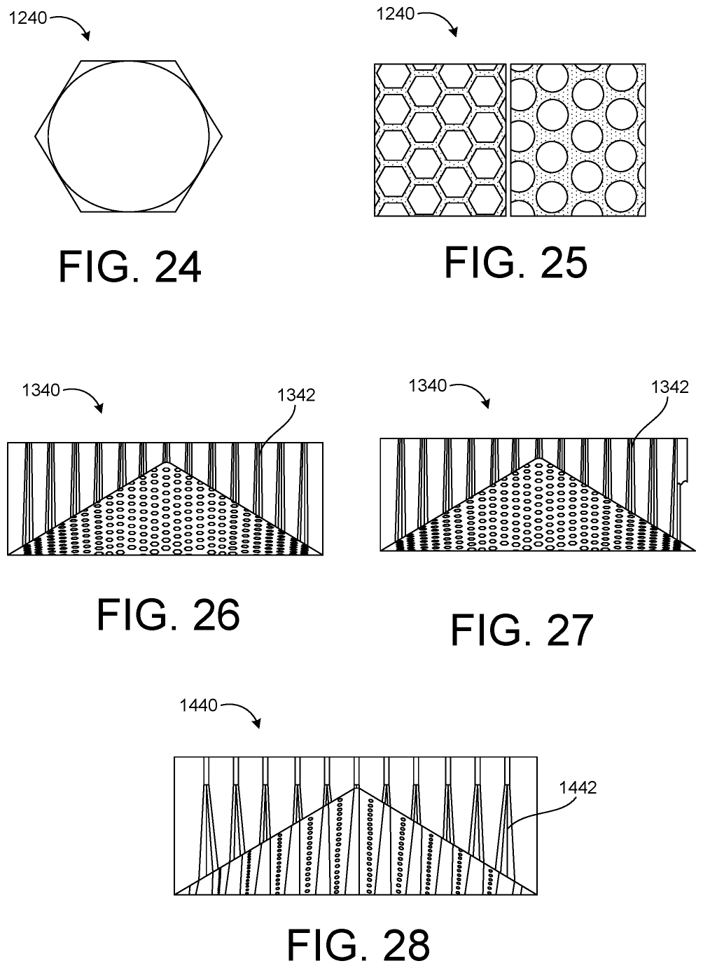

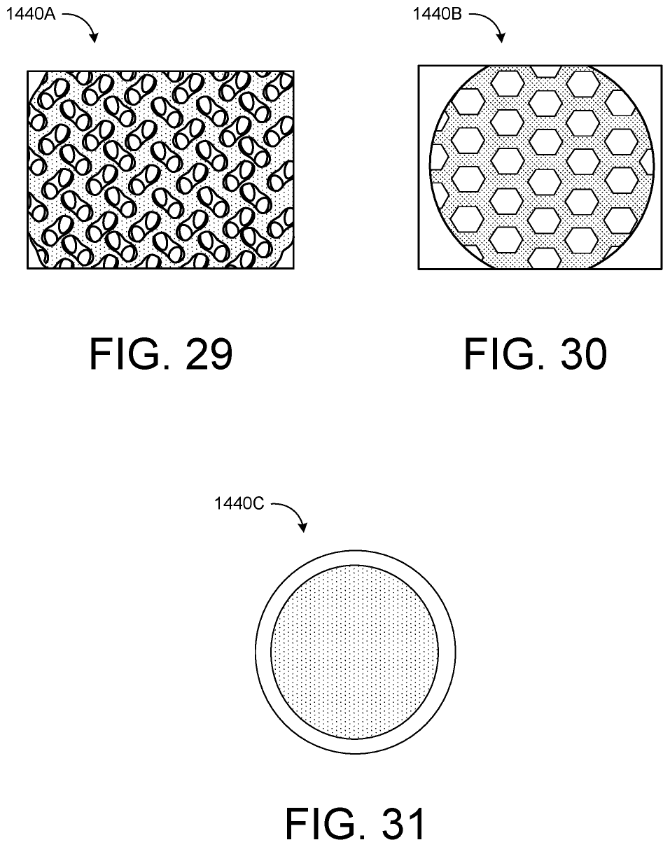

FIGS. 24-31 illustrate filters according to different embodiments. As illustrated in FIGS. 24-25, the filter 1240 may have different shaped openings. As illustrated in FIG. 26, the filter 1240 defines lumens 1242 that are linear. As illustrated in FIG. 27, the filter 1340 defines conical or tapered lumens 1342. As illustrated in FIG. 28, the filter 1440 defines Y-shaped lumens 1442. In some embodiments, the filters or flow modifiers are configured to modify or restrict the flow by blocking the flow, causing turbulence, or via another method. FIGS. 29-31 illustrate filters 1440A, 1440B, and 1440C that include openings or lumens of different shapes. In some embodiments, the filters may enhance the flow, for example by inducing laminar flow or via another method. As discussed below with respect to FIGS. 35-37, the flow may vary through the lumen.

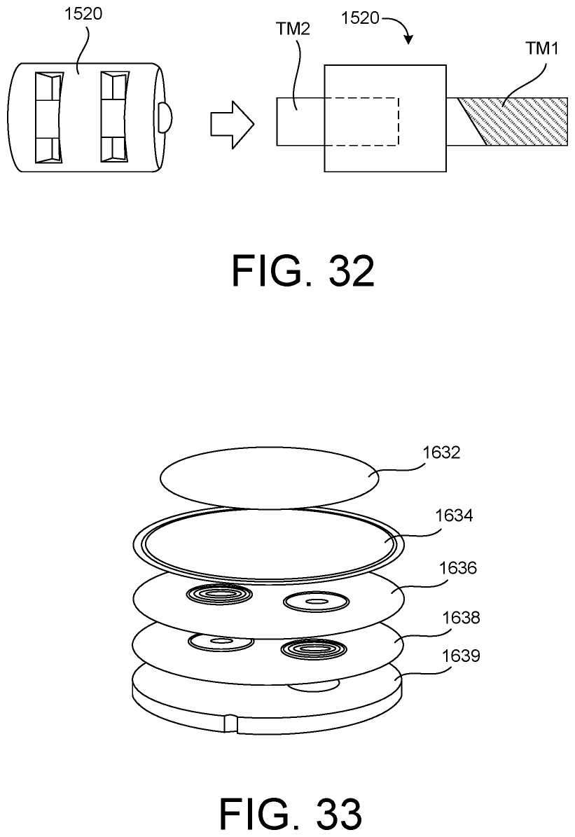

FIG. 32 illustrates a connector 1520. The connector 1520 may be used to couple tubular members together. In some embodiments, the connector 1520 may be a quick connect and may be configured to receive a portion of a first tubular member TM1 and a portion of a second tubular member TM2 to couple the first tubular member to the second tubular member.

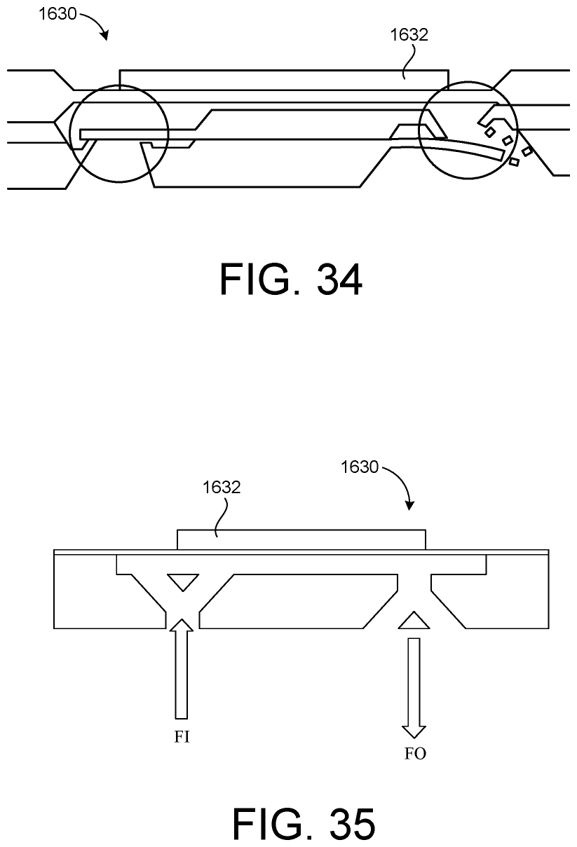

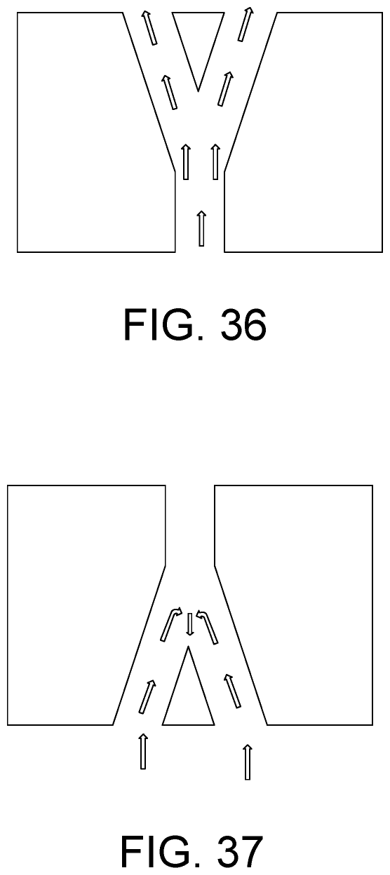

FIGS. 33 and 34 illustrate an electric pump 1630 according to an embodiment. FIGS. 35-37 illustrate fluid flow within the electric pump. Specifically, FIG. 35 illustrates the flow in FI and the flow out FO of the pump. FIG. 36 illustrates the flow through the inlet. FIG. 37 illustrates the flow through the outlet.

As illustrated in FIG. 33, the electric pump is a piezo electric pump. The pump includes a piezo 1632, a diaphragm 1634, an inlet check valve foil 1636, an outlet check valve foil 1638, and a base plate 1639. In the illustrated embodiment, the diaphragm 1634, the inlet check valve foil 1636, the outlet check valve foil 1638, and the base plate 1639 are formed of titanium. In other embodiments, such components are formed of a different material.

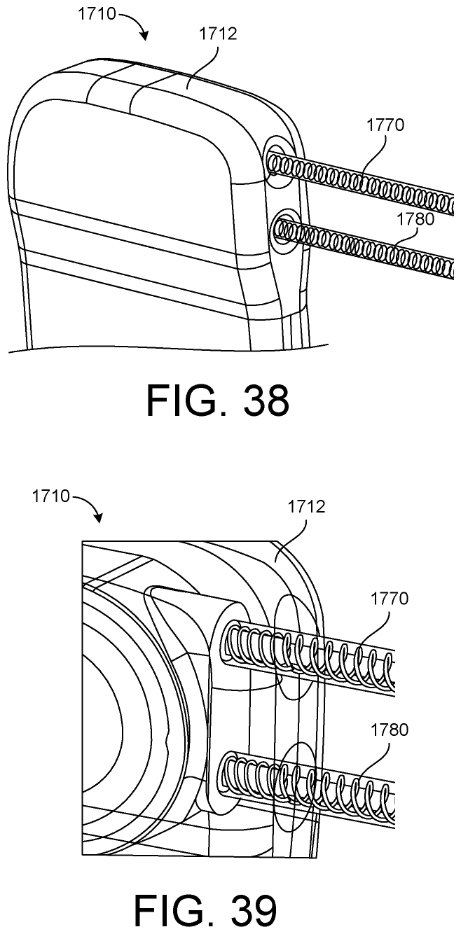

FIGS. 38-40 illustrate a pump assembly 1710 and tubular members 1770 and 1780. The pump assembly 1710 includes a housing 1712 having a top portion 1714 and a bottom portion 1716. The housing 1712 defines a cavity 1718.

In the illustrated embodiment, a coupler 1720 couples or helps couple the tubular member 1770 to the housing 1712. Coupler 1750 couples or helps couple the tubular member 1780 to the housing 1712. In the illustrated embodiment, a portion of coupler 1720 is disposed within a lumen of tubular member 1770. Similarly, a portion of coupler 1750 is disposed within a lumen of tubular member 1780.

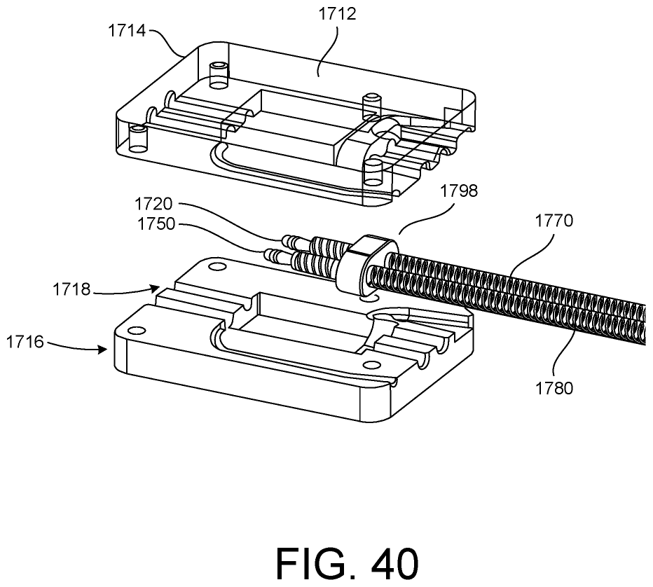

As best illustrated in FIG. 40, a form member 1798 is coupled to tubular members 1770 and 1780. The form member 1798 is disposed within the cavity 1718 of the housing 1712. The form member 1798 is configured to engage inner portions of the housing 1712 to give support to the tubular members 1770 and 1780. In some embodiments, the form member 1798 is formed of silicone. In other embodiments, the form member 1798 is formed of a different material.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implant, comprising:
an inflatable member;
a pump assembly, the pump assembly being configured to facilitate a transfer of fluid to and from the inflatable member;
a tubular member extending between the inflatable member and the pump assembly; and
a coupling member, the coupling member being configured to facilitate a coupling of the tubular member to the pump assembly.

2. The implant of claim 1, wherein the coupling member includes a first portion disposed within a lumen defined by the tubular member and a second portion disposed within a housing of the pump assembly.

3. The implant of claim 1, wherein the coupling member includes a first portion disposed within a lumen defined by the tubular member, the first portion of the coupling member having a projection member.

4. The implant of claim 1, wherein the coupling member includes a first portion disposed within a lumen defined by the tubular member, the first portion of the coupling member having a barb member.

5. The implant of claim 1, wherein the pump assembly includes a housing member having a projection, the projection of the housing member being disposed within a lumen defined by the tubular member, the coupling member including a spring biased clip, the coupling member being disposed such that a portion of the tubular member is disposed between the spring biased clip and a portion of the projection.

6. The implant of claim 1, wherein the pump assembly includes a housing member having a projection, the projection of the housing member being disposed within a lumen defined by the tubular member, the coupling member includes an overlapping wire member.

7. The implant of claim 1, wherein the pump assembly includes a housing member having a projection, the projection of the housing member being disposed within a lumen defined by the tubular member, the coupling member includes an overlapping wire member, the overlapping wire member including a screw member.

8. The implant of claim 1, wherein the coupling member includes a first portion disposed within a lumen defined by the tubular member and a second portion disposed within a housing of the pump assembly, the coupling member defining a lumen and having a filter disposed within the lumen defined by the coupling member, the filter being configured to modify a flow of fluid through the lumen.

9. The implant of claim 1, wherein the coupling member includes a first portion disposed within a lumen defined by the tubular member and a second portion disposed within a housing of the pump assembly, the coupling member defining a lumen and having a filter disposed within the lumen defined by the coupling member, the filter being a gyroid filter.

10. The implant of claim 1, wherein the coupling member includes a first portion disposed within a lumen defined by the tubular member and a second portion disposed within a housing of the pump assembly, the coupling member defining a lumen and having a filter disposed within the lumen defined by the coupling member, the first portion of the coupling member being laser welded to the housing of the pump assembly.

11. The implant of claim 1, wherein the coupling member includes a first portion disposed within a lumen defined by the tubular member, the implant further comprising:
a form member disposed such that a portion of the tubular member is disposed between the form member and a portion of the coupling member.

12. The implant of claim 1, wherein the coupling member includes a first portion disposed within a lumen defined by the tubular member, the implant further comprising:
a form member disposed such that a portion of the tubular member is disposed between the form member and a portion of the coupling member, the form member being disposed within a housing of the pump assembly.

13. The implant of claim 1, wherein the tubular member is a first tubular member, the implant further comprising:
a second tubular member; and
a form member defining a first lumen and a second lumen, the first tubular member extending through the first lumen of the form member, the second tubular member extending through the second lumen of the form member.

14. The implant of claim 1, wherein the tubular member is a first tubular member, the implant further comprising:
a second tubular member; and
a form member defining a first lumen and a second lumen, the first tubular member extending through the first lumen of the form member, the second tubular member extending through the second lumen of the form member, the form member being disposed within a housing of the pump assembly.

15. The implant of claim 1, wherein the tubular member is a first tubular member, the implant further comprising:
a second tubular member; and
a form member defining a first lumen and a second lumen, the first tubular member extending through the first lumen of the form member, the second tubular member extending through the second lumen of the form member, the form member being disposed such that a portion of the first tubular member is disposed between the coupling member and the form member.

16. An implant, comprising:
an inflatable member;
a reservoir configured to hold fluid;
a pump assembly, the pump assembly being configured to facilitate a transfer of the fluid to and from the inflatable member;
a first tubular member extending between the inflatable member and the pump assembly;
a first coupling member, the first coupling member being configured to facilitate a coupling of the first tubular member to the pump assembly;
a second tubular member extending between the reservoir and the pump assembly; and
a second coupling member, the second coupling member being configured to facilitate a coupling of the second tubular member to the pump assembly.

17. The implant of claim 16, wherein the first coupling member includes a first portion disposed within a lumen defined by the first tubular member and a second portion disposed within a housing of the pump assembly.

18. The implant of claim 16, wherein the first coupling member includes a first portion disposed within a lumen defined by the first tubular member, the first portion of the first coupling member having a projection member.

19. The implant of claim 16, wherein the first coupling member includes a first portion disposed within a lumen defined by the first tubular member, the first portion of the first coupling member having a barb member.

20. The implant of claim 16, wherein the pump assembly includes a housing member having a projection, the projection of the housing member being disposed within a lumen defined by the first tubular member, the first coupling member including a spring biased clip, the first coupling member being disposed such that a portion of the first tubular member is disposed between the spring biased clip and a portion of the projection.
